(19) United States
(12) Patent Application Publication
(10) Pub. No.: US 2022/0218717 A1
(43) Pub. Date: Jul. 14, 2022

(54) PROCESS FOR MANUFACTURING SOFT CHEWABLE FREE FLOWING GRANULES AND COMPANION ANIMAL PRODUCTS THEREOF

(71) Applicant: CUCKOS PHARMACEUTICAL PRIVATE LIMITED, Haryana (IN)

(72) Inventors: Vishal OHLAN, New Delhi (IN); Bharat Bhushan SANDUJA, Haryana (IN)

(21) Appl. No.: 17/595,636

(22) PCT Filed: May 17, 2020
(86) PCT No.: PCT/IN2020/050445

§ 371 Date: Nov. 19, 2021

(30) Foreign Application Priority Data
May 21, 2019 (IN) 201911020006

(51) Int. Cl. A61K 31/5395; A61K 9/00; A61K 9/20; A61K 31/496

(52) U.S. Cl. CPC A61K 31/5395; A61K 9/0056; A61K 9/2077; A61K 31/496; A61K 9/2095

(57) ABSTRACT

The present invention relates to soft chewable composition and its method of manufacturing using pharmaceutical conventional equipment. The present invention relates to process for manufacturing soft chewable dosage form by manufacturing free flowing soft chewable granules and these granules are compressed to make soft chewable tablets using rotary compression machine. The free-flowing granules of the invention are formed of active ingredients or nutritional agents, diluents, binder, disintegrant, sugar component, oil component or humectant or combination of both, flavour, lubricant or plasticizers in intragranular or extra granular and other conventional tableting aids to help in making the tablet more palatable.

### FIELD OF INVENTION

The Present invention relates to composition and method of manufacturing palatable soft chewable tablets or granules for delivering companion animal pharmaceutical active agent or nutritional supplement. This palatable soft chewable veterinary composition is manufactured by rotary compression from free flowing granules. Synergism between ingredients used and manufacturing method gives fast production rate of soft chewable tablets or granules with uniform weight, shape and texture without forming soft chew mass during manufacturing.

### BACKGROUND OF INVENTION

Chewable pharmaceutical dosage units, such as soft chewable tablets or chews, are known and have been commercialized for animals since long such as Interceptor Plus, Sentinel Spectrum, Quillin, and many other products. A preferred alternative dosage form for use especially with animals is the edible soft chewable dosage form. Formulation of a drug into such a soft chewable tablet, granule or other dosage form can increase animal acceptance of the medication, especially companion animals, which tend to resist swallowing hard tablets or capsules.

EP 0891776 relates to granular composition comprising simethicone and granular anhydrous calcium phosphate.

U.S. Pat. No. 8,293,265 product and process of manufacturing soft chewable medication vehicle for delivery of pharmaceutical active ingredient by forming a soft chewable mixture having meat-like texture followed by molding of the said mixture into individual unit masses.

U.S. Pat. No. 6,387,381 discloses soft chewable pharmaceutical dosage units in which an extrudate is formed of a matrix having starch, Sugar, fat, polyhydric alcohol and water.

U.S. App. No. 20190022013 and U.S. Pat. No. 10,117,831 discloses product and process of manufacturing an edible soft-chewable dosage form by forming a granulated softchew mass.

Soft chewable tablets are typically manufactured by molding and extrusion. The heat generated during the molding and extrusion process can cause deterioration in the stability (potency or integrity) of the active in the mixture, causing the effective dose provided by each unit formed to vary. Further such processes are complicated and lengthy unit operation. Additionally, conventional tablet compression techniques by formulation of a soft chew mass are also used to form soft tablets.

The Present inventors found that technology used to manufacture soft chewable tablets by compression is not a feasible process because of difficulty in making soft free flowing chewable granules from soft chewable mass. Granules formed from soft chewable mass are not free flowing and again it make soft chewable mass during manufacturing step like mixing and compression due to sticky and soft in nature. Milling of such mass poses great challenges in manufacturing.

The aforementioned problems with respect to formation of soft chewable mass are not addressed thus far. Hence there is a growing need for alternate ways to formulate compositions for a method of manufacture for edible soft chewable medications manufactured from conventional technology which can give fast production rate of soft chewable tablets with uniform weight, shape and texture and avoid one or more of the disadvantages associated with existing chewable formulations manufacturing methods and at the same time palatable to animals.

The present inventors has addressed this problem and found surprising synergy between ingredients used and manufacturing process as described in the present invention.

### SUMMARY OF INVENTION

The present invention relates to manufacturing of free-flowing soft chewable granules using pharmaceutical conventional equipment and these granules are compressed to make soft chewable tablets using rotary compression machine. The free-flowing granules of the invention is formed of active ingredients or nutritional agents, diluents, binder, disintegrant, sugar component, oil component or humectant or combination of both, flavour and plasticizers in intragranular or extra granular and other conventional tableting aids to help in making the tablet more palatable.

In one aspect, the present invention relates to a unique edible soft chewable tablet composition and processes for its manufacture. The edible soft chewable tablet of the invention is particularly palatable to companion animals.

In an aspect, the soft-chewable composition for the oral administration of a pharmaceutical active agent to a human or animal, comprising pharmaceutical active agent or nutritional supplement or combination thereof, wheat germ and other pharmaceutically or veterinary acceptable excipients.

In another aspect, the present invention relates to process for manufacturing soft chewable dosage form by manufacturing free flowing soft chewable granules. Steps of manufacturing free flowing granules consist of manufacturing wet soft granules comprising, blending of wet soft granules and fillers and sifting of blends using conventional sifter. These free-flowing soft chewable granules are blended with extra granular material and finally compressed to manufacture palatable, edible, soft chewable tablets using rotary compression machine.

Further, in another aspect, these soft chewable granules can be directly given to companion animal by sprinkling onto the companion animal food.

In a particular aspect, the present invention provides soft chewable free flowing granules composed of companion animal pharmaceutical active agent or nutritional supplement, wheat germ and oil and/or humectants, sweetener, flavor and other conventional excipients in making palatable, edible soft chewable tablets.

### DETAIL DESCRIPTION OF INVENTION

The present invention relates processes for its manufacturing of soft chewable dosage form made from free-flowing soft chewable granules which can be easily compressed by using conventional equipment and rotary compression machine.

The term “soft chewable” dosage form is a solid pharmaceutical dosage form at room temperature that has hardness less than about 2 kp, which is used to characterize a product that is not as hard and crunchy. Dosage forms of the present invention are selected from tablet, sachets or other solid forms. Preferable dosage form is soft chewable tablet or granules.

The term “Drug” refers to any therapeutic compound, or molecule, or therapeutic active agent, pharmaceutical active agent or biologically active compound.

The term “about” refers to any value which lies within the defined range by present inventors from a variation of up to ±10% of the claimed value.

The term “Composition” refers to pharmaceutical composition, wherein drug is delivered via oral route. For example, the dosage forms for composition includes tablets, granules or sachets.

The term “Wheat Germ” refers to wheat germs and the various milling by-products of these like defatted wheat germ, wheat feed flour, wheat germ with corn syrup solids, wheat middling, mixed feed, wheat shorts, wheat red dog.

The present invention is edible, palatable soft chewable dosage form with companion animal pharmaceutical active ingredient or nutritional supplements.

In particular, the present invention of soft chewable free flowing granules is composed of companion animal pharmaceutical active agent or nutritional supplement, wheat germ and oil and/or humectants, sweetener, flavor and other excipients in making palatable, edible soft chewable tablets.

One embodiment relates to composition of palatable free-flowing soft chewable granules comprising companion animal pharmaceutical active agent or nutritional supplement, wheat germ, oil component, humectant, plasticizer, sweetener, flavour, binder, disintegrant, diluents and other conventional tableting aids to help in making the soft chewable tablets.

In one embodiment, companion animal pharmaceutical active or nutritional supplement is selected from various treatment groups, non-limiting examples like antibiotics, analgesics, antiviral, antifungal, anti-parasitic, hormones, anti-inflammatory (including nonsteroidal anti-inflammatory), steroids, benzimidazole derivatives, anthelmintic, antiemetic, janus kinase inhibitor, antihypertensive, selective serotonin reuptake inhibitors, behavior modifiers, anticonvulsants, anxiolytics, hypnotics, sedatives, tranquilizers, appetite stimulants, cardiovascular agents, cardiac inodilator, insecticide, commonly companion animal supplements such as minerals and vitamins along with other nutraceutical agents, joint supplements, skin care supplements, hair care supplements, digestive supplements, dental supplements, or any combination of any two or more thereof. Active ingredients can be a single active ingredient, or a mixture of two or more active ingredients. Active ingredients given below are nonlimiting examples.

As a non-limiting example, the pharmaceutically active agent is antibiotics such as beta-lactams, penicillinase resistant antibiotics, extended spectrum antibiotics, cephalosporins, monobactams, quinolones, chloramphenicols, tetracyclines, macrolides, lincosamides, aminoglycosides, sulfonamides, glycopeptides, fluoroquinolones, topoisomerase inhibitors, antifolates or any combination of any two or more thereof.

In particular embodiment, antibiotics are selected from, cephalosporins, cephamycins, trimethoprim, dimetridazoles, erythromycin, framycetin, fruazolidone, various pleuromutilins such as thiamulin, valnemulin, various macrolides, streptomycin, clopidol, salinomycin, monensin, halofuginone, narasin, robenidine, quinolones, preferably fluoroquinolones or any combination of any two or more thereof.

In an embodiment, fluoroquinolones antibiotics are selected from benofloxacin, binfloxacin, cinoxacin, ciprofloxacin, danofloxacin, difloxacin, enoxacin, enrofloxacin, fleroxacin, ibafloxacin, levofloxacin, lomefloxacin, marbofloxacin, moxifloxacin, norfloxacin, ofloxacin, orbifloxacin, perfloxacin, temafioxacin, tosufloxacin, sarafloxacin, and sparfloxacin. Specific examples of other quinolones include pipemidic acid, nalidixic acidor any combination of any two or more thereof. In an embodiment, betalactams antibiotics are selected from clavulanic acid, sulbactam, tazobactam or any combination of any two or more thereof. In an embodiment, penicillinase resistant antibiotics are selected from methicillin, nafcillin, oxacillin, aminopenicillins, ampicillin or any combination of any two or more thereof. In an embodiment, cephalosporins are selected from cephalothin, cefazolin, cephapririn, cephalexin, cefacor, cefotetanor any combination of any two or more thereof. In an embodiment, monobactamsare selected from aztreonam or the like. In an embodiment, lincosamidesare selected from clindamycin or any same class drug. In an embodiment, macrolidesare selected from erythromycin, azithromycin, clarithromycin. In an embodiment, tetracyclinesare selected from tetracycline, doxycycline, minocycline, demeclocyclineor any combination of any two or more thereof. In an embodiment, aminoglycosides are selected from amikacin, gentamicin, kanamycin, neomycin, and tobramycin or any combination of any two or more thereof.

In preferable embodiment, antibiotics are selected from orbifloxacin, enrofloxacin, marbofloxacin, amoxiclav or the like. Examples of antibiotics are incorporated for reference, which are non limiting.

In an embodiment, insecticides can be selected from a variety of well-known different chemical classes such as chlorinated hydrocarbons, organophosphates, carbamates, pyrethroids, formamidines, borates, phenylpyrazoles, and macrocytic lactones. In one embodiment insecticides are spinosad, imidacloprid, fenthion, fipronil, allethrin, resmethrin, fenvalerate, permetrin, malathion and derivatives thereof. According to another embodiment insecticides are those of the neonicotinoid class, for example acetamiprid, clothianidin, dinotefuran, imidacloprid, nitenpyram, thiacloprid and thiamethoxam. Widely used insect growth regulators (IGRs) are, for example benzoylphenylureas such as diflubenzuron, lufenuron, noviflumuron, hexaflumuron, triflumuron, and teflubenzuron or substances like fenoxycarb, pyriproxifen, methoprene, kinoprene, bydroprene, cyromazine, buprofezin, pymetrozine and derivatives thereof. According to preferable embodiment, insecticides of the present invention are selected fromnitenpyram, lufenuron, spinosad or any combination of any two or more thereof.

In an embodiment, anthelmintics are selected from endo-parasiticides and endecticides including any of the following well-known groups such as macrocyclic lactones, benzimidazoles, pro-benzimidazoles, imidazothiazoies, tetrahydropyrimidines, organophosphates, piperazines, salicylanilide, and cyclic depsipeptides. As per other embodimentsanthelmintics comprise broad spectrum macrocyclic lactones, such as avermectins, milbemycins and derivatives thereof, including ivermectin, doramectin, moxidectin, selamectin, emamectin, eprinomectin, milbemectin, abamectin, milbemycinoxime, nemadectin, and derivatives thereof, in free form or in the form of a pharmaceutical acceptable salt. Benzimidazoles, benzimidazolecarbamate and probenzimidazoles include potent compounds such as thiabendazole, mebendazole, fenbendazole, oxfendazole, oxibendazole, albendazole, luxabendazole, netobimin, parbendazole, flubendazole, cyclobendazole, febantel, thiophanate and derivatives thereof, imidazothiazoles include highly active compounds such as tetramisole, levamisole, and derivatives thereof. Tetrahydropyrimidines include highly active compounds such as morantel, pyrantel, and derivatives thereof. Organophosphates include potent compounds such as dichlorvos, haloxon, trichlorfon, and derivatives thereof. Salicylanilides include highly active compounds such as closantel, tribromsalan, dibromsalan, oxychlozanide, clioxanide, rafoxanide, brotianide, bromoxanide and derivatives thereof. Cyclic depsipeptides include compounds consisting of amino acids and hydroxycarboxylic acids as ring structural units and 8 to 30 ring atoms, such as PF 1022A, emodepside or combination of two or more. According to preferable embodiment, anthelmintic of the present invention is moxidectin.

In an embodiment, pharmaceutically active agent is Non-Steroidal Anti-inflammatory agents. NSAIDs include, but are not limited to enolic acid and carboxylic acid derivatives. Enolic acid NSAIDs include, but are not limited to, pyrazolones (e.g. phenylbutazone, oxyphenbutazone, and ramifenazone) and oxicams (e.g. meloxicam, piroxicam, and tenoxicam). Carboxylic acid NSAIDs include, but are not limited to, salicylates (e.g. aspirin), propionic acids (e.g. ibuprofen, naproxen, carprofen, ketoprofen, and vedaprofen), anthranilic acids (e.g. tolfenamic and meclofenamic acids), phenylacetic acids (e.g. acetaminophen), aminonicotinic acids (e.g. flunixin), and indolines (e.g. indomethacin). Further non-limiting examples of NSAIDs include acemetacin, acetylsalicylic acid (aspirin), alminoprofen, benoxaprofen, bucloxic acid, celecoxib, clidanac, Robenacoxib, deracoxib, diclofenac, diflunisal, dipyrone, etodolac, fenoprofen, fentiazac, firocoxib, flobufen, flufenamic acid, flufenisal, flunixin, fluprofen, flurbiprofen, ibuprofen, indomethacin, indoprofen, isoxicam, ketoprofen, ketorolac, meclofenamic acid, mefenamic acid, miroprofen, nabumetone, naproxen, niflumic acid, oxaprozin, oxepinac, phenylbutazone, piroxicam, pirprofen, pramoprofen, sudoxicam, sulindac, suprofen, tepoxalin, tiaprofenic acid, tiopinac, tolfenamic acid, tolmetin, trioxaprofen, zidometacin, or zomepirac, pharmaceutically acceptable salts thereof and Combination thereof. According to preferable embodiment, NSAIDS are selected from robenacoxib, deracoxib, carprofen, firocoxib, meloxicam or combination thereof.

In another embodiment, the pharmaceutically active agent is antiparasitics. Nonlimiting examples of antiparasitics include macrocyclic lactones such as abamectin, ivermectin, eprinomectin, doramectin, moxidectin, selamectin, milbemycinoxime. Other examples include Prazequantel, Milbemycin, PyrantelPamoate, Afoxolaner, Sarolaner, Lotilaner. The parasiticidal agent may be an endoparasiticidal agent, ectoparaciticidal agent, orendectoparaciticidal agent. Ectoparasiticides include, for example, organochlorines, organophosphates, carbamates, amidines, pyrethrins and synthetic pyrethroids, benzoylureas, juvenile hormone analogues, macrocyclic lactones, neonicotinoids, phenylpyrazoles, and spinosyns, such as spinosad. Endectoparaciticides include, for example, macrocyclic lactones, such as ivermectin. Endoparasiticides include, for example, anthelmintics, such as those described herein. Examples of parasiticidal agents include avermectin, milbemycin, phenylpyrazole, nodulisporic acid, clorsulon, closantel, quinacrine, chloroquine, vidarabine, nitenpyram, ivermectin, milbemycineoxime, lufenuron, salimectin, moxidectin, dorimectin, and paraherquamide. According to preferable embodiment, Anti-Parasitics are selected from prazequantel, milbemycin, pyrantelpamoate, afoxolaner, sarolaner, lotilaner or combination thereof.

In another embodiment, the pharmaceutically active agent is cardiac Inodilator. Non limiting examples include phosphodiesterase inhibitors like amrinone, milrinone, enoximone and vesnarinone, beta receptor agonists like dopamine, dobutamine, epinephrine, isroterenol and xamoterol, growth hormone somatotropin, calcium sensitizing agents like pimobendan and levosimendan, myosin activators like omecamtivemecarbil and other agents like digoxine, istaroxime or combination thereof. According to preferable embodiment pimobendanis selected as cardiac inodialotor. According to another embodiment cardiac vascular agents include but not limited to, lepirudin, alteplase, reteplase, anistreplase, tenecteplase, desmopressin, vasopressin, streptokinase, felypressin, valsartan or any drugs of such class.

In one embodiment, the pharmaceutically active agent is an antiemetic. Non-limiting examples of antiemetics include neurokinin-1 (NK1) receptor antagonist such as maropitant, phenothiazines such as prochloperazine, promethazine, thiethylperazine, perphenazine, chlorpromazine, metopimazine, acepromazine, 5HT-3 receptor antagonists such as ondansetron, granisetron, tropisetron, dolasetron, hydrodolasetron, azasetron, ramosetron, lerisetron, indisetron and palonosetron, and others such as dimenhydrinate, diphenhydramine (which can also act as an antihistamine), cyclizine, meclizine, promethazine, hyroxyzine, metoclopramide, domperidone, hyoscine, hyoscine hydrobromide, hyoscine hydrochloride, scopolamine, clebopride, alizapride, itopride, bromopride, droperidol, haloperidol, benzquinamide, cerium oxalate, diphenidol, dronabinol, nabilone, ginger, levosulpiride, butorphanol and aprepitant. According to preferable embodiment antiemetic is Maropitant.

In one embodiment, the pharmaceutically active agent is janus kinase inhibitor. nonlimiting examples of janus kinase inhibitor include oclacitinib, ruxolitinib, tofacitinib, baricitinib, filgotinib, cerdulatinib, gandotinib, lestaurtinib, momelotinib, pacritinib, upadacitinib, peficitinib, fedratinib, cucurbitacin i, tofacitinib or combination thereof. According to preferable embodiment Janus Kinase Inhibitor is Oclacitinib.

In one embodiment, the pharmaceutically active agents are antihypertensive. nonlimiting examples of antihypertensive include diuretics such as loop diuretics (bumetanide, ethacrynic acid, furosemide, torsemide), thiazide diuretics (epitizide, hydrochlorothiazide and chlorothiazide, bendroflumethiazide, methyclothiazide, polythiazide), thiazide-like diuretics (indapamide, chlorthalidone, metalozone, xipamide, clopamide), potassium-sparing diuretics (amiloride, triamterene, spironolactone, eplerenone), calcium channel blockers such as amlodipine, cilnidipine, clevidipine, felodipine, isradipine. lercanidipine, levamlodipine, nicardipine, nifedipine, nimodipine, nisoldipine, nitrendipine, ace inhibitors such as captopril, enalapril, fosinopril, lisinopril, moexipril, perindopril, quinapril, ramipril, trandolapril, benazepril, angiotensin ii receptor antagonists such as azilsartan, candesartan, eprosartan, irbesartan, losartan, olmesartan, telmisartan, valsartan, fimasartan, adrenergic receptor antagonists, vasodilators, renin inhibitors, aldosterone receptor antagonists, alpha-2 adrenergic receptor agonists, endothelium receptor blockers. According to preferable embodiment antihypertensive is benazepril hydrochloride. In another embodiment Alpha-2 adrenergic receptor agonists used in present invention also act as anxiety reducing, sedative, and pain medication. In a preferred embodiment such anxiety reducing, sedative, and pain medication is dexmedetomidine.

In one embodiment, the pharmaceutically active agents are Selective Serotonin Reuptake Inhibitor. Non-limiting examples of Selective Serotonin Reuptake Inhibitor include citalopram, escitalopram, fluoxetine, fluvoxamine, paroxetine, sertraline, dapoxetine or combination thereof or the like. According to preferable embodiment selective Serotonin Reuptake Inhibitor is Fluoxetine.

In one embodiment, the pharmaceutically active agents are benzimidazoles or derivatives thereof, non-limiting examples of benzimidazole derivatives include thiabendazole, mebendazole, fenbendazole, oxfendazole, oxibendazole, albendazole, luxabendazole, netobimin, parbendazole, flubendazole, cyclobendazole, febantel, thiophanate and derivatives thereof. According to preferable embodiment benzimidazole derivatives is fenbendazole.

In one embodiment, the pharmaceutically active agents are tyrosine kinase inhibitors. Non-limiting examples of tyrosine kinase inhibitor are toceranib, sorafenib, dasatinib, sunitinib, nilotinib, gefitinib, erlotinib, imatinib, rebosutinib, lapatinib, pazopanib, and regorafenib or the like. According to preferable embodiment tyrosine kinase inhibitor is toceranib.

In one embodiment nutritional ingredient includes any ingredient whose purpose is to assist or maintain the health of the target animal. Non-limiting example, the nutritional active could be a vitamin. Non-limiting examples of vitamins include vitamin A, vitamin E, vitamin B12, vitamin B3, d-pantothenic acid (vitamin B5), folic acid, vitamin B6, vitamin B1, vitamin D3, vitamin C, vitamin B2. As another example, the nutritional active could be a pro-vitamin, for example beta-carotene or panthenol. As another non-limiting example, the nutritional active could be a mineral. Non-limiting examples of minerals include potassium, sodium, manganese, zinc, iron, calcium, copper, cobalt, iodine, chlorine and selenium. The mineral may be in the form of a suitable salt. As another example, the nutritional active could be a glycosaminoglycan or a nutritionally active monomer thereof. As a non-limiting example, the glycosaminoglycan could be chondroitin. As a non-limiting example, the glycosaminoglycan monomer could be glucosamine. As another example, the nutritional active could be an amino acid. Suitable amino acids include but are not limited to the 20 naturally occurring L-amino acids, for example arginine, isoleucine, leucine, lysine, etc. As another example, the nutritional active could be a co-enzyme, for example coenzyme Q, Vitamin, pro-vitamin, mineral, glycosaminoglycan or a nutritionally active monomer thereof, an amino acid, or a co-enzyme.

The soft chewable tablet of the present invention contains about 0.0001% to about 50% of active ingredients or nutritional agent of the soft chewable. In various embodiments, the soft chewable contains active ingredients in amount from about 0.01 wt % to about 40 wt %, from about 0.1 wt % to about 35 wt %, from about 1 wt % to about 30 wt %, from about 5 wt % to about 30 wt %, or from about 10 wt % to about 30 wt %. Preferable amount of active ingredients or nutritional agent is 0.03 to 30%.

The amount of active ingredient depends on the active ingredient(s), the animal being treated, the state of condition, and the severity of the conditions. The determination of those factors is well within the level of one skilled in the veterinary arts.

The soft-chewable composition for the oral administration of a pharmaceutical active agent to a human or animal, comprising pharmaceutical active agent or nutritional supplement or combination thereof, wheat Germ and other pharmaceutically or veterinary acceptable excipients.

The composition described herein comprise one or more pharmaceutically or veterinary acceptable excipients. The excipients are any pharmaceutically or veterinary acceptable to formulate soft chewable tablet or granules, Non-limiting examples such as diluents, oil component, humectant, sweetener, flavor, plasticizer, binder, disintegrant or combination of any two or more. In some embodiments a single excipient has more than one function in the formulation of the present invention. Excipients given below are non-limiting examples.

The Diluents or fillers are selected from, but not limited to, manitol, lactose, an inorganic phosphates like dibasic calcium phosphate, cellulose and their derivatives like microcrystalline cellulose or powdered celluloses alone or in combination with Guar gum, starches and their derivatives like hydrogenated starch hydrosylate, protein matrices like soy protein, dextrates, wheat gluten, whey, corn cob, corn meal/gluten, carbohydrates like maltodextrin, sugars and sugar alcohols such as glucose, lactose, fructose, maltose, dextrose, sucrose, maltitol, xylitol, isomalt, mannitol, polydextrose, silicates, calcium sulfate, dextrates, kaolin, magnesium carbonate, polymethacrylates, talc, or salts (e.g. sodium chloride), polyhydric alcohols and other similar saccharides, oligomers and polymers, any combination of any two or more thereof. In other embodiment diluent or filler are cereal grains and meals or flours obtained upon grinding cereal grains such as wheat germ, corn, oats, wheat, milo, barley, rice, and the various milling by-products of these cereal grains such as defatted wheat germ, wheat feed flour, wheat middling, mixed feed, wheat shorts, wheat red dog, oat, hominy feed, and other such material. Preferred diluent or fillers are wheat germ, microcrystalline cellulose, guar gum, dibasic calcium Phosphate or combination thereof. Diluents or Fillers are present in an amount from about 2%-80% by weight of composition, preferably from about 5-60% by weight of composition. In particular embodiment, wheat germ is used in amount of from about 10%-40% of the composition. In particular embodiment amount of Avicel CE 15 (microcrystalline cellulose and Guar gum) is from about 5%-25% by weight of composition. In particular embodiment amount of dibasic calcium phosphate is from about 2% to 30%. In some embodiments diluents has more than one function in the formulation of the present invention such as sweetner.

The oil components are selected from, but not limited to, fat or fats, both natural and synthetic. Oil employed as an ingredient in the soft chew may be a saturated or unsaturated liquid fatty acid, its glyceride derivatives or fatty acid derivatives of plant or animal origin or a mixture thereof. A Source for typical animal fats or oils are fish oil, chicken fat, tallow, choice white grease, prime steam lard and mixtures thereof. However, other animal fats are also suitable for use in the soft chew. Suitable sources for vegetable fats or oils can be derived palm oil, palm hydrogenated oil, corn germ hydrogenated oil, castor hydrogenated oil, cotton-seed oil, soybean oil, castor oil, olive oil, peanut oil, palm oleic oil, Cacao fat, margarine, butter, shortening and palm oil, and mixtures thereof. Additionally, a mixture of animal or vegetable oils or fats is suitable for use in the matrix. Preferable oil component is soyabean oil, castor oil or the like. Oil component is present in an amount from about 1% to 30%, preferably from about 2%-20%.

The humectants are selected from, but not limited to, sodium and potassium chloride, benzalkonium chloride, aluminium silicate, sodium propionates, sodium and potassium phosphates, sugars, sulfites, hydrogenated starch hydrosylate, etc. Liquid humectants include, but are not limited to, glycols, polyols, sugar alcohols, vegetable oils and mineral oil, hydrogenated vegetable oils, hydrocarbons, triacetin, liquid paraffin, or any combination of any two or more thereof. Other suitable humectants include glycerol, glycerol triacetate, glycerol monostearate, propylene glycol, cetyl alcohol, polydextrose, and lactic acid. Preferably humectants are selected from glycerin or the like. Humectants are present in an amount from about 1%-40% by weight of composition, preferably from about 2%-25% by weight of composition.

Palatability enhancers improve the taste of material that is chewed. Advantageously, palatability enhancers improve the palatability of soft-chewable formulations comprising bitter, acrid, obnoxious, unpleasant, or otherwise unpalatable nutritional or pharmaceutically active agents.

In one embodiment, the palatability enhancer is a taste masking agent, a flavoring agent, an aroma modifier, or a taste modifier, or any combination of any two or more thereof. Flavoring agents are used to improve the palatability of the chewable tablets by improving either its taste and/or smell. The use of a flavoring agent also assists with dose compliance.

The flavors, include, but are not limited to an artificial flavoring agent, semisynthetic flavoruring agent, a natural flavoring agent, or nature identical flavoring agent. Flavouring agent is selected from fruit, meat (e.g. pork, chicken, beef, fish), vegetable, dairy, honey, or plant derived, or is artificial. In one embodiment, the flavouring agent is selected from beef flavour, artificial beef type flavour, beef mince, pork liver powder, cheese flavour, roast chicken hickory smoke, stewed beef, chicken fat, savoury flavouring, roast pork, lamb, fish flavouring, liver, milk, cheese and egg, vanilla, creamy vanilla, butter caramel, peppermint, sweet apple, or any combination of any two or more thereof. In one embodiment the flavouring agent is selected from artificial beef type flavour, beef mince, pork liver powder, cheese flavour, roast chicken hickory smoke, or any combination of any two or more thereof. Other flavourings include plant proteins, such as soy protein, yeasts, or lactose to which edible artificial food-like flavourings has been added. Depending on the target animal, other nonanimal flavourings could include anise oil, carob, peanuts, fruit flavours, herbs such as parsley, celery leaves, peppermint, spearmint, garlic, or combinations thereof. The flavour enhancer may be a sweetener. Thus, the flavour enhancer may comprise a sugar substitute or another flavour. Preferably flavours are artificial powdered flavor, Yeast flavour, Pork flavor, Provesta or the like. The Flavours are present in an amount ranging from about 5% to about 35% by weight of the composition, preferably from about 10%-25% by weight of composition.

The Sweeteners are selected from, but not limited to, sugar, confectionary sugar, honey, maple syrup, sugar alcohols. Further, the use of the term “sugar” shall include a “sugar substitute” or an “artificial sweetener”. The sugar component may comprise white sugar, invert sugar, corn syrup, sorbitol, mannitol, oligosaccharide, isomalto oligosaccharide, fructose, lactose, glucose, lycasin, xylitol, lactitol, erythritol, mannitol, isomaltose, polydextrose, raffinose, dextrin, galactose, dextrose, saccharose, sucralose, sucrose, glycerol, hydrogenated starch hydrolysates or any mixture thereof. Articial sweeteners including saccharin, sodium saccharin, aspartame, sodium cyclamate, cyclamate salts, free cyclamic acid, dihydrochalcones, Laspartyl-L-phenylalanine methyl ester, isomaltitol, Acesulfame-K are used or any combination of any two or more thereof. Other sweetners include molasses, polyhydric alcohols and other similar saccharides oligomers and polymers and mixture thereof. Preferred Sweetener is confectionary sugar. Sweetener is present in an amount from about 1% to 30%, preferably from about 2%-20%.

The Plasticizers, include, but are not limited to alcohols, glycols (such as propylene glycol), lanolin, wool fat, liquid paraffin, mineral oil, petrolatum, benzyl phenylformate, chlorobutanol, glycerol, propylene glycol, sorbitol, triacetin, benzyl phenyl formate, PLGA polyethylene glycol, methacrylates, phthalates, acetyltributyl citrate, acetyltriethyl citrate, castor oil, dibutyl sebacate, tributyl citrate, triethyl citrate, acetyl tributyl citrate, acetyl triethyl citrate, acetylated fatty acid glycerides, castor oil, diethyl phthalate, diethyl sebacate, dibutylsebacate, dimethyl phthalate, glycerylmonostearate, glyceryl triacetate, polyoxyethylene/polyoxypropylene copolymers, dibutyl phthalate, oils, or any combination of any two or more thereof and the like. Preferable plasticizer is polyethylene glycol. The plasticizer is present in an amount ranging from about 0.5% to about 50% by weight of the composition.

The binders include, but are not limited to cellulose, methyl cellulose, polyethylene glycol, alginate, molasses, corn syrup, peanut butter, a starch such as potato starch, tapioca starch or corn starch, honey, maple syrup, sugars, gum arabic, pectins, modified starches, alginates, carrageenans, xanthan gums, carboxymethylcellulose, hydroxyethylcellulose, hydroxypropylcellulose, hydroxypropylmethylcellulose, propylene glycol alginate, polyvinylpyrrolidone (PVP), carboxyvinyl polymers (such as Carbopol. RTM.), polyethylene oxide polymers (such as Polyox.RTM.), talc, dicalcium phosphate, saccharides, Proteins, synthetic polymers, antacids or any combination of any two or more thereof and the like. Preferable binder includes polyvinylpyrrolidone K30. The binders are present in an amount ranging from about 0.5% to about 25% by weight of the composition.

The disintegrants include, but are not limited to, crosslinked polymers such as crosslinked polyvinylpyrrolidone (crospovidone), crosslinked sodium carboxymethyl cellulose (croscarmellose sodium), modified starch such as sodium starch glycolate, celluloses and their derivatives, starches and their derivatives, gelatin, alginic acid, silicon dioxide, or any combination of any two or more thereof. Preferable disintegrant is croscarmellose or combination of other disintegrants or the like. The disintegrants are present in an amount ranging from about 0.5% to about 25% by weight of the composition.

The Lubricant includes, but is not limited to magnesium stearate, fumaric acid, sodium stearyl fumarate, talc, silica, fats, polyethelene glycol and sodium pamoate. Lubricants are present in an amount from about 2% —about 20% by weight of composition.

The present invention relates to process for manufacturing free flowing soft chewable granules by manufacturing soft wet granules, blending of wet soft granules with fillers to make free flowing granules and sifting of free-flowing granules using conventional sifter.

In particular, the present invention relates to process for manufacturing granules of soft chewable pharmaceutical composition comprising:
a. manufacturing soft wet granules from active agent or nutritional supplement, wheat germ and other pharmaceutically or veterinary acceptable excipients,
b. blending of wet soft granules with fillers, and
c. sifting of granules using conventional sifter;

Wherein granules obtained in said process are free flowing.

These free-flowing soft chewable granules are blended with or without extra granular material and finally compressed to manufacture palatable, edible, soft chewable tablets using rotary compression machine.

The present invention specifically relates to method for manufacturing soft chewable dosage forms using compression process, wherein liquid components such as soybean oil and glycerin absorbed into wheat germ to form wet granules. Wet granules are blended with diluents which enable to produce free flow granules. These free-flowing granules are sifted through conventional sifter. These free-flowing granules are easily compressible and no problem of compressibility, sticking, lamination during compaction process and gives fast production rate of soft chewable tablets with uniform weight and texture.

In another embodiment, the present invention relates to manufacturing of Soft free flowing granules which can be easily compressed into soft chewable tablets by using conventional equipment like RMG for granules manufacturing and rotary compression machine for soft chewable tablet manufacturing.

According to another embodiment, the pharmaceutical composition is manufactured by a process comprising the steps of
1. Dry mixing of Drug, fillers or diluents, flavor, and dry binder,
2. Granulation by adding humectants or oil or both,
3. Blending of wet granules and filler,
4. Sifting of granules of step 3 and mixing of extra granular material,
5. Compression of granules using rotary compression machine.

Wherein extra granular material comprises the ingredients incorporated before the compression of tablet which are not contained in the granules, which optionally comprising of active agent or nutritional agent, fillers or diluents, disintegrant, falvours, sweetners, lubricant or other excipients. In another embodiment, a pharmaceutical active agent or nutritional supplement or combination thereof is used as intragranular with other excipients.

The Flow of the powder during manufacturing dictates the quality of the product in terms of its weight and content uniformity. Flow of granules affects manufacturing efficiency. The free-flowing properties are advantageous for the preparation of solid formulations especially for soft chewable tablet.

The present invention relates to soft-chewable composition comprising pharmaceutical active agent or nutritional supplement or combination thereof, wherein free-flowing granules are obtained during manufacturing.

The granules in present invention having a bulk density and tapped density to give fair, good or excellent flow characteristics. The Hausner ratio ranges from about 1.00—about 1.25 and Compressibility index ranges from about 1%—about 20% for the granules of the present invention.

The inventors have found surprisingly that wheat germ is having role in absorbing liquid components of composition and hence aids to produce free flow material along with other inactive ingredients. Liquid components such as soyabean oil, glycerin or any other liquid components are absorbed into wheat germ during mixing, which enables to produce free flowing granules along with other inactive ingredient like anhydrous dibasic calcium phosphate. These free-flowing granules are easily compressible and resolves problem of compressibility, sticking, lamination during compaction process and gives fast production rate of soft chewable tablets with uniform weight and texture.

In an embodiment, process for manufacturing granules comprising following steps:
1. Dry mixing a pharmaceutical active agent or nutritional supplement, wheat germ, flavor, and dry binder,
2. Granulating the mixture obtained in step 1 by adding humectants or oil or both,
3. Blending the wet granules obtained in step 1 with diluents or fillers,
4. Sifting the granules of step 3 and mixing extra granular material,

Wherein granules obtained in said process are free flowing and allows favorable flow property for compression and does not allow formation of the sticky mass during process.

In most preferred embodiment, process of manufacturing of soft chewable dosage forms prepared from free flowing granules comprising the steps of
1. Dry mixing of Drug, Wheat germ, flavor, sweetener & dry binder,
2. Granulation by adding soybean oil and humectants such as Glycerin,
3. Blending of wet granules and anhydrous Dibasic Calcium Phosphate,
4. Sifting of granules of step 3 and mixing of extra granular material,
5. Compression of granules using rotary compression machine.

Important embodiment of the present invention offers free flowing granules by manufacturing wet granules in RMG without milling and mixing with the extra granular part in Non-shear type blender. The inventors have found that dry binders have good ability to bind dry powders and undergo direct compaction. Addition of oil and humectants such as glycerin in the RMG is the critical parameter. One important aspect relates to absorbing humectant into the excipients like wheat germ by means of encapsulating humectant inside the granules and providing dry coat of excipient by mixing another excipient to make them free flowing. Thus humectant is used to make the granules and later excipient is blended with the granules to make them free flowable. Prepared free flowing granules can be filled into hopper to make soft chewable tablets like conventional tablet compression process. Soft chewable tablets manufactured from conventional technology are easy to manufacture with consistent in weight, shape and texture.

The following examples are given for the purpose of illustrating the present invention and should not be considered as limitation on the scope or spirit of the invention.

### Example 1 Soft Chewable Tablet Composition

TABLE 1

| Ingredients | Concentration |
|---|---|
| Antibiotic | 0.5-8% |
| Wheat Germ | 25-30% |
| Flavor | 10-25% |
| MCC and Guargum coprocessed | 5-10% |
| Confectioner Sugar | 5-10% |
| Soyabean Oil | 3-10% |
| Glycerin | 10-20% |
| Croscarmellose sodium | 5-10% |
| Dibasic Calcium Phosphate | 10-20% |
| Polyethylene Glycol | 5-10% |
| Total Tablet weight | 3000 mg |

Process of manufacturing soft chewable tablet
1. Dry mixing of an antibiotic with Wheat germ, Flavor (natural and/or artificial), microcrystalline cellulose with guar gum (co-processed) and confectionary sugar and Croscarmellose sodium,
2. Granulation by adding Soybean oil and Glycerin,
3. Blending of wet granules and addition of dibasic calcium phosphate to make the granules free flowing,
4. Sifting of granules of step 3 through suitable sieve and mixing of extra granular material (rest of the %) in non-shear type blender,
5. Compression of final blend using rotary compression machine at defined parameters using specific punches.

### Example 2 Soft Chewable Tablet Composition

TABLE 2

| Ingredients | Concentration |
|---|---|
| NSAID (Non steroidal | 0.5-5% |
| anti-inflammatory Drug) | |
| Wheat Germ | 25-30% |
| Flavor | 10-25% |
| MCC and Guargum coprocessed | 5-10% |
| Confectioner Sugar | 5-10% |
| Soyabean Oil | 3-10% |
| Glycerin | 10-20% |
| Croscarmellose sodium | 5-10% |
| Dibasic Calcium Phosphate | 10-20% |
| Polyethylene Glycol | 5-10% |
| Total Tablet weight | 3000 mg |

Process of manufacturing soft chewable tablet
1. Dry mixing of NSAID with Wheat germ, Flavor (natural and/or artificial), microcrystalline cellulose with guar gum (co-processed) and confectionary sugar and Croscarmellose sodium,
2. Granulation by adding Soybean oil and Glycerin,
3. Blending of wet granules and addition of dibasic calcium phosphate to make the granules free flowing,
4. Sifting of granules of step 3 through suitable sieve and mixing of extra granular material (rest of the %) in non shear type blender,
5. Compression of final blend using rotary compression machine at defined parameters using specific punches.

### Example 3 Soft Chewable Tablet Composition

TABLE 3

| Ingredients | Concentration |
|---|---|
| Cardiac Inodilator | 0.04-5.0% |
| Wheat Germ | 25-30% |
| Flavor | 10-25% |
| MCC and Guargum co processed | 5-10% |
| Confectioner Sugar | 5-10% |
| Soyabean Oil | 3-10% |
| Glycerin | 10-20% |
| Croscarmellose sodium | 5-10% |
| Dibasic Calcium Phosphate | 10-20% |
| Polyethylene Glycol | 5-10% |
| Total Tablet weight | 3000 |

Process of manufacturing soft chewable tablet
1. Dry mixing of a Cardiac inodilator, wheat germ, flavor (natural and/or artificial), microcrystalline cellulose with guar gum (co-processed), confectionary sugar and Croscarmellose sodium,
2. Granulation by adding Soybean oil and Glycerin,
3. Blending of wet granules and addition of dibasic calcium phosphate to make the granules free flowing,
4. Sifting of granules of step 3 through suitable sieve and mixing of extra granular material (rest of the %) in non shear type blender,
5. Compression of final blend using rotary compression machine at defined parameters using specific punches.

### Example 4 Soft Chewable Tablet Composition

TABLE 4

| Ingredients | Concentration |
|---|---|
| Anti-parasitic agent | 0.08-45% |
| Wheat Germ | 25-30% |
| Flavor | 10-25% |
| MCC and Guargum co processed | 5-10% |
| Confectioner Sugar | 5-10% |
| Soyabean Oil | 3-10% |
| Glycerin | 10-20% |
| Croscarmellose sodium | 5-10% |
| Dibasic Calcium Phosphate | 10-20% |
| Polyethylene Glycol | 5-10% |
| Total Tablet weight | 3000 |

Process of manufacturing soft chewable tablet
1. Dry mixing of an Anti-parasitic agent, Wheat germ, Flavor (natural and/or artificial), microcrystalline cellulose with guar gum (co-processed), confectionary sugar and Croscarmellose sodium,
2. Granulation by adding Soybean oil and Glycerin,
3. Blending of wet granules and addition of dibasic calcium phosphate to make the granules free flowing,
4. Sifting of granules of step 3 through suitable sieve and mixing of extra granular material (rest of the %) in non shear type blender,
5. Compression of final blend using rotary compression machine at defined parameters using specific punches.

### Example 5 Exemplary Method of Manufacture for Soft Chewable Tablet

Sift all the ingredients listed in example 1-4 through #30 sieve using vibrosifter, or alternatively can be done using co mill at medium speed. Load the material into RMG and granulate with oil and humectant. Unload the material from RMG and mix in non-shear type blender, add extra granular portion and mix in non-shear type blender. Compress the final blend at specified parameters using the specified punches.

### Example 6 Enrofloxacin Soft Chewable Granules

TABLE 5

| | | |
|---|---|---|
| INGREDIENTS | mg/tab | percentage |

INTRAGRANULAR

| | | |
|---|---|---|
| Wheat Germ | 540 | 20.77 |
| Flavor | 260 | 10.00 |
| Microcrystalline Cellulose + | 200 | 7.69 |
| Guar Gum | | |
| Croscarmellose sodium | 100 | 3.85 |
| Confectionary Sugar | 90 | 3.46 |
| Soyabean Oil | 64 | 2.46 |
| Glycerin | 340 | 13.08 |

EXTRAGRANULAR

| | | |
|---|---|---|
| Enrofloxacin | 136 | 5.23 |
| Dibasic Calcium Phosphate | 300 | 11.54 |
| Flavor | 260 | 10.00 |
| Sodium Starch Glycolate | 100 | 3.85 |
| Polyethylene Glycol | 210 | 8.08 |
| TOTAL WEIGHT | 2600 | 100 |

Process of manufacturing soft chewable granules
1. Dispensing each ingredient separately,
2. Sifting Wheat germ, Flavor (natural and/or artificial), microcrystalline cellulose with guar gum (co-processed), Crosscarmellose Sodium and confectionary sugar through a suitable sieve and loading the sifted material in Rapid Mixer Granulator (RMG),
3. Dry mixing step 2 material at a specified impeller and chopper speed for 10 min in RMG,
4. Granulating step 3 material by addition of Soybean oil and Glycerin into RMG,
5. Withdrawing wet granules from RMG and adding directly to the blender along with the sifted extragranular material (Enrofloxacin, Dibasic calcium Phosphate, Flavor, Sodium Starch Glycolate and Polyethylene Glycol) in a stepwise manner,
6. Blending step 5 material at a definite speed for 20 minutes,
7. Withdrawing the step 6 material and sift the material through a suitable sieve,
8. Loading the sifted step 7 materials in Fluidized bed dryer and fluidizing the material to attain a certain temperature and maintain at the same temperature for 10-15 minutes,
9. Withdrawing the blend and spreading on the different trays to allow the blend to cool and attain room temperature.

### Example 7 Marbofloxacin Soft Chewable Tablet

TABLE 6

| | | |
|---|---|---|
| | Example 7 | |
| INGREDIENTS | mg/tab | Percentage |

INTRAGRANULAR

| | | |
|---|---|---|
| Marbofloxacin | 200 | 7.69 |
| Wheat Germ | 475 | 18.27 |
| Flavor | 260 | 10.00 |
| Microcrystalline Cellulose + | 200 | 7.69 |
| Guar Gum | | |
| Croscarmellose sodium | 80 | 3.08 |
| Confectionary Sugar | 90 | 3.46 |
| Soyabean Oil | 65 | 2.50 |
| Glycerin | 410 | 15.77 |

EXTRAGRANULAR

| | | |
|---|---|---|
| dibasic calcium Phosphate | 200 | 7.69 |
| Flavor | 260 | 10.00 |
| Sodium Starch Glycolate | 100 | 3.85 |
| Polyethylene Glycol | 260 | 10.00 |
| TOTAL WEIGHT | 2600 | 100.00 |

Process of manufacturing soft chewable tablet
1. Dry mixing of Marbofloxacin, Wheat germ, Flavor (natural and/or artificial), microcrystalline cellulose with guar gum (co-processed), confectionary sugar and Croscarmellose sodium,
2. Granulation by adding Soybean oil and Glycerin,
3. Withdrawing wet granules from RMG and adding directly to the blender along with the sifted extragranular material (Dibasic calcium Phosphate, Flavor, Sodium Starch Glycolate and Polyethylene Glycol) in a stepwise manner,
4. Blending step 3 material at a definite speed for 20 minutes,
5. Withdrawing the step 4 material and sift the material through a suitable sieve,
6. Loading the sifted step 5 materials in Fluidized bed dryer and fluidizing the material to attain a certain temperature and maintain at the same temperature for 10-15 minutes,
7. Withdrawing the blend and spreading on the different trays to allow the blend to cool and attain room temperature,
8. Compression of final blend using rotary compression machine at defined parameters using specific punches.

### Comparative Example 8 Marbofloxacin Soft Chewable Tablet without Use of

Wheat Germ

TABLE 7

| | | |
|---|---|---|
| | Comparative | |
| | Example 8 | |
| INGREDIENTS | mg/tab | Percentage |

INTRAGRANULAR

| | | |
|---|---|---|
| Marbofloxacin | 200 | 7.69 |
| Silicified Microcrystalline | 455 | 17.50 |
| cellulose | | |
| Flavor | 260 | 10.00 |
| MCC + Guar Gum | 220 | 8.46 |
| Crosscarmellose Sodium | 80 | 3.08 |
| Confectioner Sugar | 100 | 3.85 |
| Soyabean oil | 65 | 2.50 |
| Glycerin | 390 | 15.00 |

EXTRAGRANULAR

| | | |
|---|---|---|
| Dibasic Calcium Phosphate | 210 | 8.08 |
| Flavor | 260 | 10.00 |
| Sodium Starch Glycolate | 100 | 3.85 |
| Polyethylene Glycol | 260 | 10.00 |
| Total Weight | 2600 | 100.00 |

Process of manufacturing soft chewable tablet
1. Dry mixing of Marbofloxacin, Silicified Microcrystalline cellulose, Flavor (natural and/or artificial), microcrystalline cellulose with guar gum (co-processed), confectionary sugar and Croscarmellose sodium,
2. Granulation by adding Soybean oil and Glycerin,
3. Withdrawing wet granules from RMG and adding directly to the blender along, with the sifted extragranular material (Dibasic calcium Phosphate, Flavor, Sodium Starch Glycolate and Polyethylene Glycol) in a stepwise manner,
4. Blending step 3 material at a definite speed for 20 minutes,
5. Withdrawing the step 4 material and sift the material through a suitable sieve,
6. Loading the sifted step 5 materials in Fluidized bed dryer and fluidizing the material to attain a certain temperature and maintain at the same temperature for 10-15 minutes,
7. Withdrawing the blend and spreading on the different trays to allow the blend to cool and attain room temperature,
8. Compression of final blend using rotary compression machine at defined parameters using specific punches.

Physical parameters of the granules formed from above examples 6, 7 and comparative example 8 are mentioned in below Table 8, which shows flow property of granules.

Flow Property parameter of Granules of Composition with Wheat germ (Example 6, Example 7) and without wheat Germ (Example 8)

TABLE 8

| Parameters | Example 6 | Example 7 | Example 8 |
|---|---|---|---|
| Bulk Density | 0.46 gm/ml | 0.50 gm/ml | 0.51 gm/ml |
| Tapped density | 0.53 gm/ml | 0.62 gm/ml | 0.73 gm/ml |
| Hausner ratio | 1.15 | 1.24 | 1.43 |
| Compressibility | 13.21% | 19.35% | 30.14% |
| Index | | | |
| Flow of granules | Good | Fair | Poor |

Inventors of the current invention had found surprising effect of wheat germ in granules flow property which is supported by results shown in table 8. Granules of Example 6, Example 7 were made with use of wheat germ showed good to fair flow property of granules, while Granules of example 13 were made without wheat germ which resulted in to poor flow property of granules. From results it is very clear that Wheat germ has played a very crucial role in currently invented composition and flow property of granules.

1. A soft-chewable composition comprising, by weight of the composition, i) 0.001-50% pharmaceutically active agent or nutritional supplement or combination thereof, ii) 10-40% wheat germ and iii) one or more pharmaceutically or veterinary acceptable excipients, wherein the composition is for oral administration.

2. The soft-chewable composition as claimed in claim 1, wherein the soft-chewable composition is selected from tablet, granules, sachets or other solid dosage forms.

3. The soft-chewable composition as claimed in claim 1, wherein the wheat germ is present in an amount from about 25-30% by weight of the soft-chewable composition.

4. The soft-chewable composition as claimed in claim 1, wherein the pharmaceutically or veterinary acceptable excipients are selected from diluent or filler, oil component, humectant, plasticizer, sweetener, flavour, binder, disintegrant, and lubricant.

5. The soft-chewable composition as claimed in claim 1, wherein a pharmaceutical active agent or nutritional supplement is selected from the group consisting of antibiotics, analgesics, antiviral, antifungal, anti-parasitic, hormones, anti-inflammatory (including nonsteroidal anti-inflammatory), steroids, benzimidazole derivatives, anthelmintic, antiemetic, j anus kinase inhibitor, antihypertensive, selective serotonin reuptake inhibitors, behavior modifiers, anticonvulsants, anxiolytics, hypnotics, sedatives, tranquilizers, appetite stimulants, cardiovascular agents, cardiac inodilator, insecticide, animal supplements such as minerals and vitamins along with other nutraceutical agents, joint supplements, skin care supplements, hair care supplements, digestive supplements, and dental supplements, or any combination thereof.

6. The soft-chewable composition as claimed in claim 4, wherein the filler or diluent is selected from the group consisting of manitol, lactose, dibasic calcium phosphate, cellulose and cellulose derivatives like selected from microcrystalline cellulose or powdered celluloses, alone or in combination with Guar gum, starche and hydrogenated starch hydrosylate, protein matricese, carbohydrates, sugars and sugar alcohols selected from glucose, lactose, fructose, maltose, dextrose, sucrose, maltitol, xylitol, isomalt, mannitol, and polydextrose, dextrates, wheat gluten, whey, corn cob, corn meal/gluten, silicates, calcium sulfate, kaolin, magnesium carbonate, polymethacrylates, talc, sodium chloride, grains and meals or flours obtained upon grinding cereal grains selected from wheat germ, corn, oats, wheat, milo, barley, rice, wherein diluents or fillers are present in an amount from 2% to 80% by weight of composition.

7. The soft-chewable composition as claimed in claim 4, wherein the binder is selected from the group consisting of cellulose, methyl cellulose, polyethylene glycol, alginate, molasses, corn syrup, peanut butter, potato starch, tapioca starch, corn starch, honey, maple syrup, sugars, gum arabic, pectins, modified starches, alginates, carrageenans, xanthan gums, carboxymethylcellulose, hydroxyethylcellulose, hydroxypropylcellulose, hydroxypropylmethylcellulose, propylene glycol alginate, polyvinylpyrrolidone, polyvinylpyrrolidone K30, carboxyvinyl polymers, polyethylene oxide polymers, talc, dicalcium phosphate, saccharides, Proteins, synthetic polymers, antacids or any combination thereof; and wherein the binder is present in an amount from 0.5% to 25% by weight of composition.

8. The soft-chewable composition as claimed in claim 4, wherein the humectants are selected from the group consisting of glycerin, sodium chloride, potassium chloride, benzalkonium chloride, aluminium silicate, sodium propionates, sodium phosphates, potassium phosphates, sugars, sulfites, hydrogenated starch hydrosylate, glycols, polyols, sugar alcohols, vegetable oils, mineral oil, hydrogenated vegetable oils, hydrocarbons, triacetin, liquid paraffin, glycerol, glycerol triacetate, glycerol monostearate, propylene glycol, cetyl alcohol, polydextrose, lactic acid or any combination thereof; and wherein humectant is present in an amount from 1% to 40% by weight of composition.

9. The soft-chewable composition as claimed in claim 4, wherein the oil components is selected from the group consisting of (i) natural fat or synthetic fat, (ii) saturated or unsaturated liquid fatty acid, its glyceride derivatives or fatty acid derivatives of plant or animal origin or a mixture thereof, (iii) animal fats or oils selected from fish oil, chicken fat, tallow, choice white grease, prime steam lard or mixtures thereof, (iv) vegetable fats or oils selected from palm oil, palm hydrogenated oil, corn germ hydrogenated oil, castor hydrogenated oil, cotton-seed oil, soybean oil, castor oil, olive oil, peanut oil, palm oleic oil, Cacao fat, margarine, butter, shortening and palm oil, and mixtures thereof, or (v) mixture of animal and vegetable oils or fats; and wherein the oil component is present in an amount from 1% to 30% by weight of composition.

10. The soft-chewable composition as claimed in claim 1, wherein, the composition is free-flowing granules having Compressibility index from 1% to 20% and Hausner ratio ranges from 1.00 to 1.25.

11. A process for manufacturing granules of soft chewable pharmaceutical composition comprising:
a. manufacturing soft wet granules from pharmaceutical active agent or nutritional supplement or combination thereof, wheat germ and other pharmaceutically or veterinary acceptable excipients,
b. blending of wet soft granules with fillers, and
c. sifting of granules using conventional sifter;
wherein granules obtained in said process are free flowing.

12. The process as claimed in claim 11, comprising following steps:
a. dry mixing a pharmaceutical active agent or nutritional supplement or combination thereof, wheat germ, and other pharmaceutically or veterinary acceptable excipients,
b. granulating the mixture obtained in step a by adding humectants or oil component or both,
c. withdrawing wet granules from RMG and adding directly to the blender along with the sifted extragranular material,
d. blending step c material at a definite speed for 20 minutes,
e. withdrawing the step d material and sift the material through a suitable sieve,
f. loading the step d material in Fluidized bed dryer and fluidize the material to attain a certain temperature and maintain at the same temperature for 10-15 minutes, and
g. withdrawing the blend and spreading on the different trays to allow the blend to cool and attain room temperature.

13. The process as claimed in claim 11, wherein granules are compressed using rotary compression machine to obtain tablet.

14. The process as claimed in claim 11, wherein a pharmaceutical active agent or nutritional supplement or combination thereof used is intragranular or extragranular.

15. The process as claimed in claim 11, wherein granulation is done by rapid mixture granulator.

16. The process as claimed in claim 11, wherein the granules have Hausner ratio ranges from 1 to 1.25.

17. The process as claimed in claim 11, wherein the granules have Compressibility index ranges from 1% to 20%.
